(19) United States
(12) Patent Application Publication
(10) Pub. No.: US 2026/0191288 A1
(43) Pub. Date: Jul. 9, 2026

(54) WIPING DEVICE FOR A HELMET VISOR

(71) Applicant: Tanya Holt, Milwaukee, WI (US)

(72) Inventor: Tanya Holt, Milwaukee, WI (US)

(21) Appl. No.: 19/015,045

(22) Filed: Jan. 9, 2025

(51) Int. Cl. A42B 3/26; A61F 9/02

(52) U.S. Cl. CPC A42B 3/26; A61F 9/029

(57) ABSTRACT

A wiping device for clearing water and other debris from a visor of a helmet includes a track and a wiper. The wiper is movable between ends of the track. Each end of the track is attachable to the helmet over the visor such that the wiper may engage and wipe the visor of water or other debris. A drive mechanism is operatively coupled to the wiper.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to wiping devices and more particularly pertains to a new wiping device for clearing water and other debris from a visor of a helmet.

### (2) Description of Related Art Including Information Disclosed Under 37 CFR 1.97 and 1.98

Precipitation creates a challenge to visibility for individuals who drive motorcycles. The prior art discloses various devices for combatting such precipitation by clearing water or other debris from a visor of a helmet. However, the prior art fails to describe such a device in which opposing ends of a track are adjustably attachable to the helmet such that a wiper may be urged along the track to clear the visor of debris.

### BRIEF SUMMARY OF THE INVENTION

An embodiment of the disclosure meets the needs presented above by generally comprising a track which extends between a first end and a second end. A wiper is mounted on the track and is movable between the first end and the second end of the track. The wiper comprises an arm and a blade. The arm is elongated between a top end and a bottom end, and the blade is coupled to and extends along the arm between the top end and the bottom end. The blade protrudes away from a central longitudinal axis of the arm. A mount assembly is coupled to the track and configured to mount the track over a visor of a helmet. A drive mechanism is operatively coupled to the wiper and is configured to move the wiper alternately toward the first end of the track and the second end of the track.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front in-use view of a wiping device according to an embodiment of the disclosure.

FIG. 2 is a front view of an embodiment of the disclosure.

FIG. 3 is a detail front view of an input of an embodiment of the disclosure taken from Window 3 in FIG. 2.

FIG. 4 is a detail side view of an input of an embodiment of the disclosure.

FIG. 5 is a top view of an embodiment of the disclosure.

FIG. 6 is a cross section view of an embodiment of the disclosure taken from Line 6-6 in FIG. 5.

FIG. 7 is a block diagram of an embodiment of the disclosure.

### DETAILED DESCRIPTION OF THE INVENTION

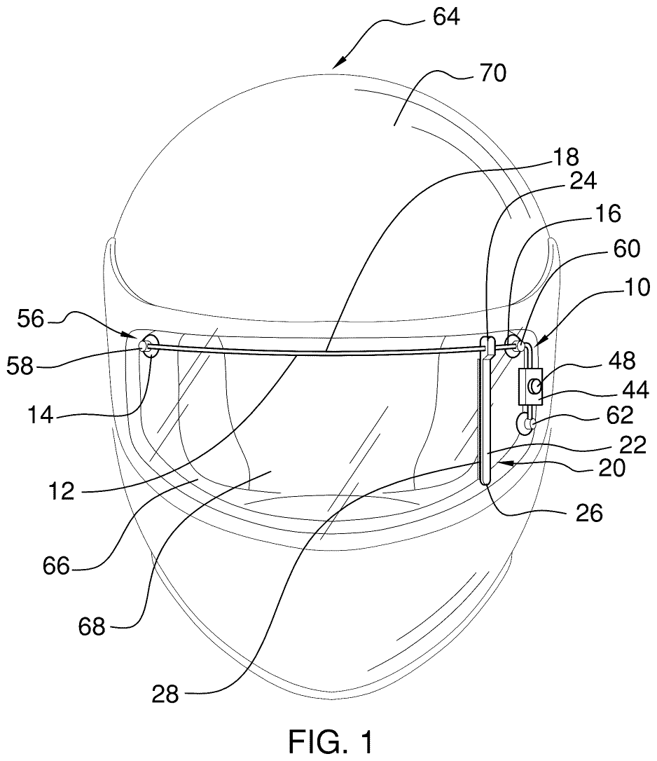

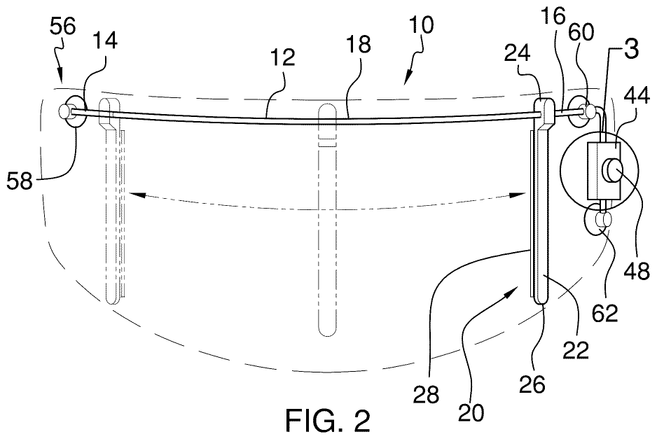

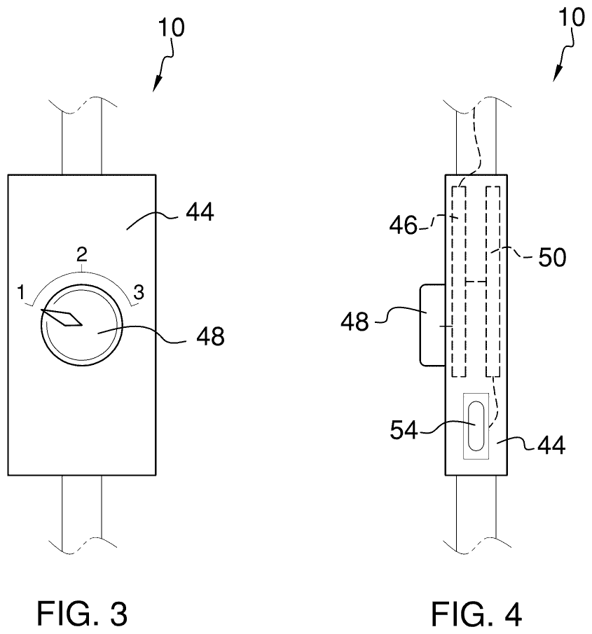

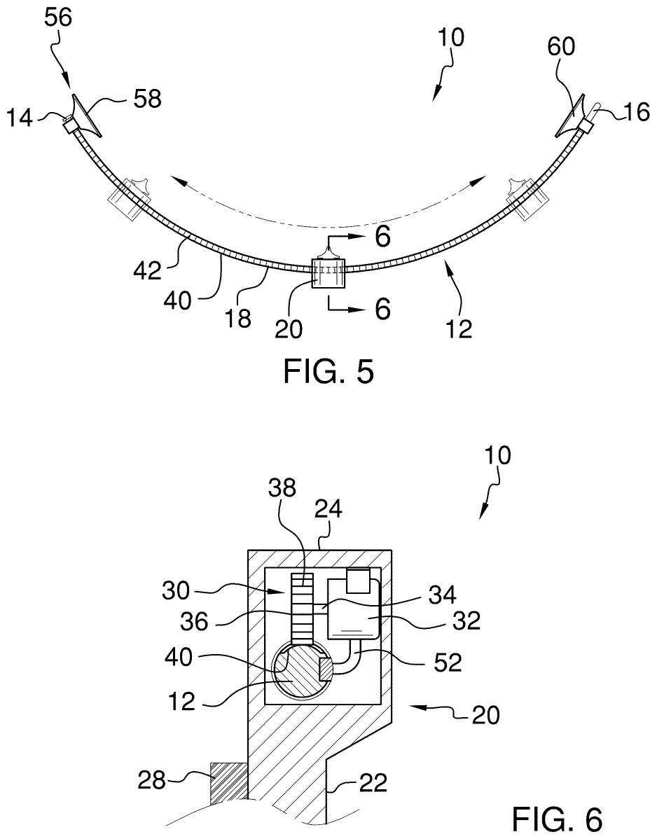

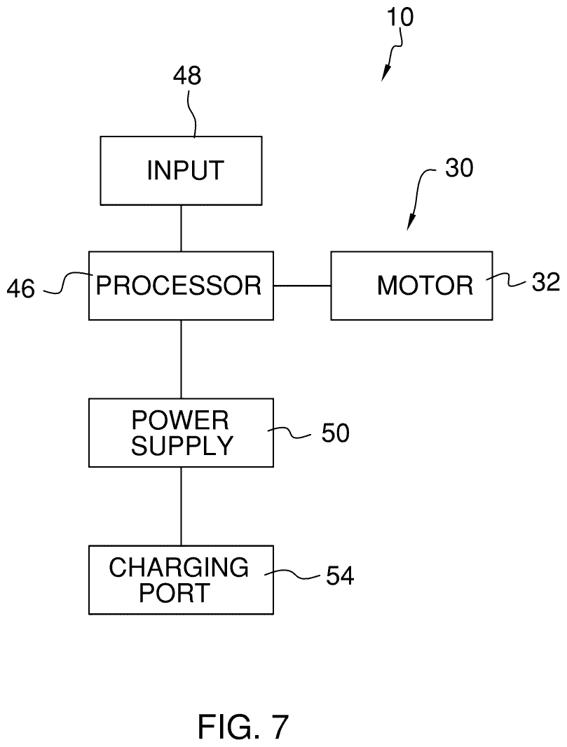

With reference now to the drawings, and in particular to FIGS. 1 through 7 thereof, a new wiping device embodying the principles and concepts of an embodiment of the disclosure and generally designated by the reference numeral 10 will be described.

As best illustrated in FIGS. 1 through 7, the wiping device 10 generally comprises a track 12 which extends between a first end 14 and a second end 16 and a wiper 20 mounted on the track 12. The wiper 20 comprises an arm 22 and a blade 28. The arm 22 is elongated between a top end 24 and a bottom end 26, and the blade 28 is coupled to and extends along the arm 22 between the top end 24 and the bottom end 26. The blade 28 protrudes away from a central longitudinal axis of the arm 22. The track 12 extends through the arm 22 of the wiper 20 adjacent to the top end 24. The blade 28 comprises a pliable material which is water impermeable such that the blade 28 may conform to a surface when drawn against it and clear water or other fluid from the surface. For example, the blade 28 may comprise rubber, silicone, or the like. The track 12 may be curved between the first and second ends 14, 16 such that the track 12 generally conforms to a front surface 68 of a visor 66 of a helmet 64. The track 12 also may comprise a bendable material such that the track 12 is bent during mounting to the helmet 64 in order to generally conform to the front surface 68 of the visor 66.

The wiper 20 is movable between the first end 14 and the second end 16 of the track 12 via a drive mechanism 30. The drive mechanism 30 comprises a motor 32, a pinion 36, and a rack 40. The motor 32 is mounted in the arm 22 of the wiper 20 adjacent to the track 12, and the pinion 36 is mounted to an output shaft 34 of the motor 32. The rack 40 is coupled to the track 12 at the top side thereof. The rack 40 has teeth 42 which mesh with gear teeth 38 of the pinion 36. The motor 32 may then be operated to rotate the pinion 36 in a first rotational direction to move the wiper 20 toward the first end 14 of the track 12 and in a second rotational direction opposite the first rotational direction to move the wiper 20 toward the second end 16 of the track 12.

A housing 44 is attached to the track 12 proximate to the second end 16 of the track 12. A processor 46 is positioned in the housing 44 and is operatively coupled to the drive mechanism 30. An input 48 is operatively coupled to the processor 46. The input 48 is actuatable to select a speed of the wiper 20. The input 48 comprises a dial which may be used to direct the processor 46 to vary the electrical power transmitted to the drive mechanism 30. In other embodiments, the input 48 may comprise one or more buttons, a touch screen, or the like. Additional sensors, switches, or the like may be employed to detect when the wiper 20 reaches the first end 14 or the second end 16 of the track 12 in order to direct the motor 32 to change the direction of rotation of the pinion 36.

A power supply 50 is mounted in the housing 44 and is electrically coupled to the processor 46. The power supply 50 comprises a battery but may comprise a capacitor or other suitable energy storage. A charging port 54 is mounted to an exterior of the housing 44 and is in electrical communication with the power supply 50. The charging port 54 may electrically couple to an external power source to charge the power supply 50. An electrical contact 52 extends along the track 12 which electrically couples the drive mechanism 30 to the processor 46, which selectively directs electricity from the power supply 50 to the drive mechanism 30. In other embodiments, the drive mechanism 30 may be electrically coupled to the processor 46 or to the power supply 50 via cables or other suitable electrical connections. In some embodiments, the drive mechanism 30 may have an independent power supply 50 and may communicate wirelessly with the processor 46.

A mount assembly 56 is coupled to the track 12 and is configured to mount the track 12 over the visor 66 of the helmet 64. The mount assembly 56 comprises a first fastener 58 positioned at the first end 14 of the track 12 and a second fastener 60 positioned at the second end 16 of the track 12. Each of the first and second fasteners 58, 60 comprises a suction cup. However, in other embodiments, the first and second fasteners 58, 60 may comprise magnets, adhesives, threaded fasteners, or other suitable fasteners. An additional fastener 62 is coupled to the housing 44 opposite the second end 16 of the track 12. The second fastener 60 and the additional fastener 62 secure opposite sides of the housing 44 to the helmet 64. The additional fastener 62 also comprises a suction cup but may comprise a magnet, adhesive, threaded fastener, or other suitable fastener.

In use, the track 12 and the housing 44 are mounted to the helmet 64 via the mount assembly 56 such that the track 12 extends over the visor 66. The first and second fasteners 58, 60 may be attached to the visor 66 or may be placed on a body 70 portion of the helmet 64. To clear water or other fluids from the visor 66, the drive mechanism 30 is operated via the input 48. When the drive mechanism 30 is activated, it moves the wiper 20 back and forth between the first end 14 and the second end 16 of the track 12, which draws a side of the blade 28 of the wiper 20 along the visor 66. The blade 28 conforms to the surface of the visor 66 and urges water away.

By excluding a fastener from a central section 18 of the track 12, a placement of the wiping device 10 on the helmet 64 may be adjusted to bring the central section 18 of the track 12 closer to or farther from the visor 66 as is desired. The first and second fasteners 58, 60 may be moved farther back on the helmet 64 to bring the central section 18 closer to the visor 66, and the first and second fasteners 58, 60 may be moved forward on the helmet 64 to move the central section 18 farther from the visor 66. Bringing the central section 18 of the track 12 closer to the visor 66 leads to better engagement of the blade 28 of the wiper 20 against a central portion of the visor 66, which is typically the area through which the wearer of the helmet 64 most needs to see. However, positioning the central section 18 of the track 12 too close can cause the visor 66 to interfere with the motion of the wiper 20.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A wiping device for a helmet, the wiping device comprising:
a track extending between a first end and a second end;
a wiper mounted on the track, the wiper being movable between the first end and the second end of the track, the wiper comprising an arm and a blade, the arm being elongated between a top end and a bottom end, the blade being coupled to and extending along the arm between the top end and the bottom end, the blade protruding away from a central longitudinal axis of the arm;
a mount assembly coupled to the track and configured to mount the track over a visor of the helmet; and
a drive mechanism operatively coupled to the wiper, the drive mechanism being configured to move the wiper alternately toward the first end of the track and the second end of the track.

2. The wiping device of claim 1, wherein the mount assembly comprises a first fastener positioned at the first end of the track and a second fastener positioned at the second end of the track.

3. The wiping device of claim 2, wherein each of the first and second fasteners comprises a suction cup.

4. The wiping device of claim 1, wherein the drive mechanism comprises:
a motor mounted in the arm of the wiper;
a pinion mounted to an output shaft of the motor; and
a rack mounted on the track, the rack having teeth which mesh with gear teeth of the pinion.

5. The wiping device of claim 1, further comprising:
a processor operatively coupled to the drive mechanism; and
an input operatively coupled to the processor, a speed of the wiper being selectable by the input.

6. The wiping device of claim 5, further comprising a power supply electrically coupled to the processor.

7. The wiping device of claim 6, wherein the power supply comprises a battery.

8. The wiping device of claim 6, further comprising a charging port electrically coupled to the power supply.

9. A wiping device for a helmet, the wiping device comprising:
a track extending between a first end and a second end;
a wiper mounted on the track, the wiper being movable between the first end and the second end of the track, the wiper comprising an arm and a blade, the arm being elongated between a top end and a bottom end, the blade being coupled to and extending along the arm between the top end and the bottom end, the blade protruding away from a central longitudinal axis of the arm;
a mount assembly coupled to the track and configured to mount the track over a visor of the helmet, the mount assembly comprising a first fastener positioned at the first end of the track and a second fastener positioned at the second end of the track, each of the first and second fasteners comprising a suction cup;
a drive mechanism operatively coupled to the wiper, the drive mechanism being configured to move the wiper alternately toward the first end of the track and the second end of the track, the drive mechanism comprising:
a motor mounted in the arm of the wiper;
a pinion mounted to an output shaft of the motor; and
a rack mounted on the track, the rack having teeth which mesh with gear teeth of the pinion;
a processor operatively coupled to the drive mechanism;
an input operatively coupled to the processor, a speed of the wiper being selectable by the input;
a power supply electrically coupled to the processor, the power supply comprising a battery; and
a charging port electrically coupled to the power supply.
